(19) United States
(12) Patent Application Publication
(10) Pub. No.: US 2004/0197341 A1
(43) Pub. Date: Oct. 7, 2004

(54) Therapeutic process for p. aeruginosa infections using macrolide antibiotics

(76) Inventors: Jean-Claude Pechere, Geneve (CH); Christian Van Delden, Cologny (CH); Oktay Menekse, Henau (CH)

Correspondence Address: FULBRIGHT & JAWORSKI, LLP, 666 FIFTH AVE, NEW YORK, NY, 10103-3198, US

(21) Appl. No.: 10/485,282

(22) PCT Filed: Sep. 3, 2001
(86) PCT No.: PCT/CH01/00532

(51) Int. Cl. A61K 39/00; A61K 39/38

(52) U.S. Cl. 424/184.1

(57) ABSTRACT

Macrolides, in particular azalides such as azithromycin, are suited for the treatment of nosocomial infections caused by P. aeruginosa. The mechanism of action is the inhibition of the quorum sensing of P. aeruginosa, in particular the impediment of the las and rhl quorum sensing systems synthesis and the impediment of the synthesis of the autoinducers N-[3-oxododecanoyl]-L-homoserine lactone and N-butyrylhomoserine lactone. This allows for treatments of P. aeruginosa infections at non-inhibiting concentrations of the macrolide.

### FIELD OF THE INVENTION

The present invention relates to the treatment or prevention of Pseudomonas aeruginosa infections.

### BACKGROUND OF THE INVENTION

P. aeruginosa, an increasingly prevalent opportunistic human pathogen, is the most common gram negative bacterium found in nosocomial (i.e. hospital-acquired) infections. P. aeruginosa is responsible for 16% of nosocomial pneumonia cases, 12% of hospital-acquired urinary tract infections, 8% of surgical wound infections, and 10% of bloodstream infections. Immunocompromised patients, such as neutropenic cancer and bone marrow transplant patients and particularly susceptible to opportunistic infections. In this group of patients, P. aeruginosa is responsible for pneumonia and septicemia with attributable deaths reaching 30%. P. aeruginosa is also one of the most common and lethal pathogens responsible for ventilator-associated pneumonia in intubated patients, with directly attributable death rates reaching 38%. P. aeruginosa bacteremia is also a source of concern in burn patients. P. aeruginosa outbreaks in burn units are associated with high (60%) death rates. In the expanding AIDS population, P. aeruginosa bacteremia is associated with 50% of deaths. Cystic fibrosis (CE) patients are characteristically susceptible to chronic infection by P. aeruginosa, which is responsible for high rates of illness and death in this population.

The capacity of P. aeruginosa to produce such infections is due to a range of extracellular virulence factors. The secretion of some extracellular virulence factors by P. aeruginosa has been shown to be controlled by two complex regulatory systems, the las and rhl quorum sensing systems (“quorum sensing” is also known as “cell-to-cell-signaling”). The principles of the las and rhl quorum sensing systems in P. Aeruginosa have been reviewed (Van Delden, C., Iglewski, B. H., Emerging Infectious Diseases, 1998, 4(4), 551-559). The first cell-to-cell signaling system described in P. aeruginosa was shown to regulate expression of the virulence factor LasB elastase and was named the las system (Passador, L., Cook, J. M., Gambello, M. J., Rust, L., Iglewski, B. H., Science 1993, 260, 1127-1130). The las cell-to-cell signaling system is composed of lasI, the autoinducer synthase gene responsible for the synthesis of 3-oxo-C₁₂-HSL (N-[3-oxododecanoyl]-L-homoserine lactone), and the lasR gene that codes for a transcriptional activator protein. The second known P. aeruginosa cell-to-cell signaling system is named the rhl system because of its ability to control the production of the virulence factor rhamnolipid. This system is composed of rhl, the C₄—HSL (N-butyrylhomoserine lactone) autoinducer synthase gene, and the rhlR gene encoding a transcriptional activator protein.

This system regulates the expression of the rhlAB operon that encodes a rhamnosyltransferase required for rhamnolipid production (Ochsner, U. A., Fiechter, A., Reiser J., J. Biol. Chem. 1994, 269, 19787-19795). The rhl system is also necessary for optimal production of LasB elastase, LasA protease, pyocyanin, cyanide, and alkaline protease.

These quorum sensing systems allows P. aeruginosa to delay the onset of production of virulence factors, in particularly of elastase and rhamnolipid, until their cell numbers have become large enough to overcome the body's immune system. The importance of quorum sensing in the pathogenesis of chronic infections, however, is unknown.

### PRIOR ART

P. aeruginosa is commonly combatted with antibiotics such as β-lactams, aminoglycosides or quinolones. Macrolide antibiotics, however, are not appreciated by the skilled person as useful in therapeutics or prevention of P. aeruginosa infections, as the minimum inhibiting concentrations (MIC's) of macrolide antibiotics for P. aeruginosa strains typically lie by a factor of 50-100 above the clinically in vivo achievable levels of macrolide antibiotics. Thus, in clinical trials of macrolide antibiotics against P. aeruginosa strains no effect on the viability of the microorganism was observed (e.g. for clarithromycin: Yanagihara, K., Tomono, K., Sawai, T., Kuroki, M., Kaneko, Y., Ohno, H., Higashima, Y., Miyazaki, Y., Hirakata, Y., Maesaki, S., Kadota, J., Tashiro, T., Kohno, S.; J. Antimicrob. Chemother. 2000, 46, 69-72; and Yanagihara, K., Tomono, K., Sawai, T., Hirakata, Y., Kadota, J., Koga, H., Tashiro, T., Kohno, S., Am. J. Respir. Crit. Care Med. 1997, 155, 337-342). Some studies have observed a benefit of longterm macrolide treatment in patients suffering from DPB or CF (for erythromycin: Fuji T., Kadota, K., Kawakami, K., Iida, R., Shirai, R., Kaseda, M., Kawamoto, S., Kohno, S., Thorax 1995, 50, 1246-1252; for azithromycin: Jaffe, A., Francis, M., Rosenthal, M., Bush, A., Lancet 351, p. 420).

A reduction of autoinducer production by 50 μg of erythromycin/ml has been suggested (Sofer, D. N., Gilboa-Garber, A., Belz, A., Garber, N. C., Chemotherapy 1999, 45, 335-341); the Chromobacterium violaceum bioassay used in this reference could only measure the C₄-HSL autoinducer, however (McClean, K. H., Winson, M. K., Fish, L., Taylor, A., Chhrabra, S. R., Camara, M., Daykin, M., Lamb, J. H., Swift, S., Bycroft, B. W., Stewart, G. S., Williams, P., Microbiology, 1997, 143, 3703-3711).

The need exists to provide a therapeutic process against P. aeruginosa infections which avoids in particularly the buildup of resistence.

### SUMMARY OF THE INVENTION

One object of the present application is an improved therapeutic process against P. aeruginosa using macrolide antibiotics, whereby the macrolide is administered in an amount which is effective in impeding quorum sensing in the said P. aeruginosa. This amount will typically be appreciably below the MIC of P. aeruginosa. In a preferred embodiment of this object, the amount is effective in impeding las or rhl quorum sensing, and particularly the amount is effective in impeding both las and rhl quorum sensing systems. The las and rhl quorum sensing systems depend on the respective autoinducer molecules 3-oxo-C₁₂-HSL (N-[3-oxodode-canoyl]-L-homoserine lactone) and C₄-HSL (N-butyrylhomoserine lactone). In a further preferred embodiment of this object, the amount of macrolide administered is therefore effective in impeding the synthesis of 3-oxo-C₁₂-HSL and/or C₄-HSL in P. aeruginosa.

In a particularly preferred embodiment of this object of the present invention the administered macrolide is an azalide, in particularly azithromycin.

A further object of the present invention is the use of a macrolide antibiotic for the manufacture of a medicament suited for combatting hospital-acquired P. aeruginosa infections, whereby the medicament contains the macrolide in an amount effective for impeding quorum sensing in P. aeruginosa. In preferred embodiment of this object, the medicament contains the macrolide in an amount effective to impede both las and rhl quorum sensing systems of P. aeruginosa; and in a particularly preferred embodiments the amount is effective for impeding the synthesis of 3-oxo-C₁₂-HSL and/or C₄-HSL in P. aeruginosa. Particularly preferred is the use of azalides (macrolides in which the macrolide ring is expanded by one nitrogen atom), in particularly azithromycin.

The inventors of the present application have found that macrolides, azalides and in particular azithromycin interfere with the quorum-sensing mechanism in P. aeruginosa. It has particularly been found that macrolides impede the las and/or rhl quorum sensing systems of P. aeruginosa, and that they inhibit the production of both autoinducer molecules C₄-HSL and 3-oxo-C₁₂-HSL essential to the quorum sensing systems of P. aeruginosa. This inhibition is achieved at concentrations much lower than the respective minimum inhibiting concentrations (MIC's) of P. aeruginosa.

### DESCRIPTION OF THE FIGURES

FIG. 1a) shows the growth of P. aeruginosa strain PAO1 (typical experiment) and its elastase and rhamnolipid production when grown in Luria-Bertani (LB) medium in the absence (squares) or in the presence of azithromycin (circles, 2 μg/ml; upside triangles, 3 μg/ml; downside triangles, 4 μg/ml; diamonds, 5 μg/ml).

FIG. 1b) shows the elastase activity (mean±standard deviation of three independent experiments performed in duplicate) of supernatants of cells, grown either in the absence (squares) or the presence (circles) of 2 μg/ml of azithromycin.

FIG. 1c) shows the expression of the rhlAB operon (in the P. aeruginosa strain PAO1 harbouring its rhlA′-lacZ reporter fusion, pECP60) when grown in LB medium either in the absence (squares) or the presence (circles) of 2 μg/ml of azithromycin (measured as β-Gal activity, mean ±standard deviation of three independent experiments performed in duplicate).

FIG. 2a) shows in strain PAO1 the expression of the lasR and rhlR genes (via lacZ reporter fusions, measured as β-Gal activities). 1, lasR without azithromycin; 2, lasR in presence of 2 μg/ml azithromycin; 3, lasR in presence of 2 μg/ml azithromycin and 3-oxo-C₁₂-HSL and C₄—HSL autoinducers (10 μM each); 4, rhlR without azithromycin; 5, rhlR in presence of 2 μg/ml azithromycin; 6, rhlR in presence of 2 μg/ml azithromycin and 3-oxo-C₁₂—HSL and C₄—HSL autoinducers (10 μM each).

FIG. 2b) shows in strain PAO1 the expression of the lasI and rhlI genes (via lacZ reporter fusions, measured as Gal activities). 1, lasI without azithromycin; 2, lasI in presence of 2 μg/ml azithromycin; 3, rhlI without azithromycin; 4, rhlI in presence of 2 μg/ml azithromycin.

FIG. 3a) shows in strain PAO1 the reduction of the production of the autoinducers 3-oxo-C₁₂-HSL and C₄-HSL. 1,3-oxo-C₁₂-HSL without azithromycin; 2,3-oxo-Cl₂-HSL in presence of 2 μg/ml azithromycin; 3, C₄-HSL without azithromycin; 4, C₄-HSL in presence of 2 μg/ml azithromycin.

FIG. 3b) shows in strain PAO1 the relative expression of the rhlAB operon, coding for rhamnosylstransferase (measured from β-Gal activities), and the production of elastase. 1, rhlAB expression without azithromycin; 2, rhlAB expression in presence of 2 μg/ml azithromycin; 3, rhlAB expression in presence of 2 μg/ml azithromycin and 3-oxo-C₁₂-HSL and C₄-HSL autoinducers (10 μM each); 4, elastase production without azithromycin; 5, elastase production in presence of 2 μg/ml azithromycin; 6, elastase production in presence of 2 μg/ml azithromycin and 3-oxo-Cl₂-HSL and C₄-HSL autoinducers (10 μM each).

### DETAILED DESCRIPTION OF THE INVENTION

The term “subject” shall mean in the context of the present application any animal, including the mammals and man.

The term “nosocomial infections” refers in the context of the present application to infections that may rise in the said subjects when they are hospitalized. Examples of such infections are pneumonia, ventilator-associated pneumonia in intubated patients, septicemia, hospital-acquired urinary tract infections following intubation with an urinary cathether, infections arising in immunocompromised (e.g. from neutropenia, AIDS) patients and cystic fibrosis.

The term “impeding” means in the context of the present application that quorum sensing, in particular the las and/or rhl quorum sensing systems, resp. the synthesis of the corresponding autoinducer molecules, is inhibited to an extent which is detectable by a suited assay.

The amount of macrolide which is effective for the treatment or prevention of the nosocomial P. aeruginosa-originated disease, by impeding quorum sensing, in particularly las and/or rhl quorum sensing and, more particularly, by impeding the synthesis of the autoinducers N-[3-oxododecanoyl]-L-homoserine lactone and/or N-butyrylhomoserine lactone in P. aeruginosa, will vary on the type of macrolide and on the P. aeruginosa strain in question and may be determined by clinical studies on laboratory animals or on human volunteers.

The primary hint that this effective in vivo amount was used, e.g. by administering the macrolide in the form of a pharmaceutical composition (see below), is the overcome of the nosocomial infection itself.

A further hint that this effective amount was achieved in vivo is the regress or absence of the symptoms associated with the P. aeruginosa infection, such as chronic inflammatory response or tissue damage, and which would follow the release of extracellular virulence factors by P. aeruginosa.

It is recalled that the eventual effect of the impediment of quorum sensing by macrolides is that the population of P. aeruginosa keeps behaving as isolated cells (i.e. the bacteria do not mutually perceive their presence anymore) and that this misleading prevents the population from producing extracellular virulence factors such as elastase and rhamnolipid.

Further experimental hints that this effective amount was achieved may be derived from assayed samples of the environment of use of the macrolide antibiotic within the subject (serum, plasma, sputum, tissue samples, smears), namely the site of the subject's body infected with P. aeruginosa. It is known that the said autoinducer molecules, essential for quorum sensing, are released by the bacteria into this environment. A comparison of samples from subjects infected with a P. aeruginosa strain, and not treated with macrolide, with samples from subjects infected with the same strain, but treated with macrolide, may reveal, at a given administered threshold amount of the macrolide, a statistically significant difference in autoinducer concentration between the samples from the two groups (statistically significant in consideration of the differences between the individual subjects of the groups and the variabilities in cell counts and behaviour of the bacterium strain). This threshold amount may then be considered as the “effective” amount. The samples may be assayed by any technique known in the art for this purpose. An example of an assay for the autoinducer 3-oxo-C₁₂-HSL may be the one described in Pearson, J. P., Pesci, E. C., Iglewski, B. H., J. Bacteriol. 1997, 179, 5756-5767; and for C₄-HSL the Chromobacterium violaceum assay (McClean, K. H., Winson, M. K., Fish, L., Taylor, A., Chhabra, S. R. Camara, M., Daykin M., Lamb, J. H., Swift, S., Bycroft, B. W., Stewart, G. S. A. B., Williams, P., Microbiology 1997, 143, 3703-3711; Shaw, P. D., Ping, G., Daly, S. L., Cha, C., Cronan, J. E. Jr., Rinehart, K. L., Farand, S. K., Proc. Natl. Acad. Sci. USA, 1997, 94, 6036-6041) or the assay described in Seed, P. C., Passador, L., Iglewski, B. H., J. Bacteriol. 1995, 177, 654-659. One example of an analysed sample is the sputum of patients suffering from cystic fibrosis (Geisenberger, O., Givskov, Riedel, K., Hoiby, N., Tummler, B., Eberl. L., FEMS Microbiol. Lett. 184, 273-278; Singh, P. K., Schaefer, A. L., Parsek, M. R., Moninger, T. O., Welsh, M. J., Greenberg, E. P., Nature 2000, 407, 762-764).

In particular the in vivo effective amount of the macrolide may be about 1 to 5 μg/ml of environment of use (e.g. serum, plasma), preferably about 1 to 3 μg/ml, and specifically about 2 μg/ml.

Exemplary macrolides that can be used in the therapeutic processes and uses according to the invention are erythromycin A and B. roxithromycin, the compound of formula (VI) of EP-B-0 699 207 and clarithromycin.

A preferred class of macrolides are the azalides, which are expanded in the macrolide ring at the C9 position by one nitrogen atom. Examples of azalides which can be used and administered according to the invention are azithromycin, the compounds (II), (III) and (IV) of EP-B-0 101 186 and the compounds (III), (V) and (VII) of EP-B-0 699 207. Particularly preferred is azithromycin.

The uses and therapeutic treatments according to the invention are suited to counteract nosocomial infections at any site within a subject's (human or animal) body which can be colonized by P. aeruginosa and which subsequently can develop symptoms of a nosocomial disease. Such a site may be considered as an environment of use of the macrolide. Examples of such environments of use are the lung tracheae and bronchiae (e.g. when incised in order to introduce an intubation for artificial respiration), superficial wound lesions, and any site of introduction of a cathether into the said body (e.g. an urinary cathether), and also the entire systemic body of the patient in cases of systemic infection such as in the case of P. aeruginosa bacteraemia.

Examples of P. aeruginosa strains that can be influenced by the therapeutic process according to the invention are all strains possessing a las and/or a rhl quorum sensing system.

Examples of such strains are ATCC 33347, PA B16, PA N42, PA103 and in particular the strain PAO1.

By the therapeutic process according to the invention the viability of the P. aeruginosa strain in question is preferably not affected by the macrolide, i.e. such treatment is non-inhibitory for P. aeruginosa. This type of treatment avoids the development of resistance in the P. aeruginosa population against the macrolide, as no selection pressure favouring macrolide-resistant strains is exerted.

The macrolides may be formulated in analogy to previously known macrolide-containing medicaments in order to carry out the processes of the invention. The amount of macrolide may be chosen such that it is effective in impeding quorum sensing in particularly the las and rhl quorum sensing systems, and specifically the synthesis of the autoinducer molecules 3-oxo-C₁₂-HSL and C₄-HSL thereof.

An example of such medicaments are oral medicaments such as tablets or capsules. It is actually only by making use of the quorum-sensing capabilities of macrolides that oral dosage forms (which typically cannot produce more than about 1.5 μg/ml macrolide serum concentration) can be employed against P. aeruginosa nosocomial infections.

The oral medicaments by which the macrolides are administered may for instance be sustained release tablets and comprise, besides the macrolide, pharmaceutically acceptable excipients and diluents common in the art. These include release-retarding or release-controlling agents such as polyethylene oxide, celluloses of varying degree of etherification such as hydroxypropyl cellulose or hydroxypropylmethlycellulose, pregelatinised starch, xanthan gum, polyvinylpyrrolidone or sodium carboxymethylcellulose, diluents such as sugars (e.g. lactose or sucrose), buffering aids such mono-, di- and tribasic phosphate salts, α-bletting aids such as glidants (e.g. magnesium stearate, sodium stearyl fumarate), and artificial flavours or colorants. The release properties of the tablets may be further influenced by special coatings such as for example an enteric coating. In the case of oral dosage forms the macrolide is preferably formulated as an once-a-day dosage form with a content of about 100 to 700 mg of the macrolide. This would correspond to a dosage of about 1.5 mg/kg to about 10 mg/kg of body weight per day (assuming 70 kg of patient's body weight). Preferably the content of the formulation is about 250 mg.

The oral medicament may also be a capsule comprising granules, pellets or beads of the macrolide. For the formulation of the pellets or granules themselves the same pharmaceutically acceptable adjuvants as with the tablets may be used.

In order to obtain an initial guess about the in vivo release of an oral macrolide formulation the in vitro release behaviour (total release vs. time) of sustained release dosage forms may be determined in a standard USP rotating paddle apparatus as disclosed in United States Pharmacopoeia XXIII (USP) Dissolution Test Chapter 711, Apparatus 2, whereby the test media may be artificial gastric or eneric juices, depending on the targeted in vivo site of release. The actually obtained in vivo concentrations of the macrolide in serum, plasma, sputum or different tissues are dependent on several factors such as type of macrolide, released concentration thereof in the stomach and/or intestine, rate of excretion thereof and affinity of the different in vivo media for the macrolide (azithromycin for instance tends to accumulate in body tissues, with rather low concentrations in serum and plasma). The determinations of in vivo concentrations following oral administration may be done by means of usual clinical trials using a representative panel of volunteers.

Medicaments for intravenous administration may be formulated as solutions in water, isotonic saline, isotonic dextrose or Ringer's solution. As the macrolides in their neutral form are sparingly soluble or even insoluble in water then optionally non-aqueous cosolvents such as dimethylsulfoxide, ethanol, glycerol, propylene glycol and other nonaqueous vehicles which will not interfere with the therapeutic efficiency of the preparation and are nontoxic in the volume or proportion used, may be admixed to the solution, in order to enhance the solubility of the active ingredient. Alternatively or in addition, the macrolides may be converted at the nitrogen atom of their desosamine moiety into an acid addition salt. The acids used here may be any pharmaceutically acceptable acid such as hydrochloric, phosphoric, sulfuric, acetic, succinic, hemisuccinic (halfesterified), tartaric, hemitartaric (half-esterified) and boric acids. The ethyl hemisuccinate of erythromycin A e.g. is marketed as-Erythro ES®. In the case of the azalides conversion into a disalt is possible. The dihydrochloride of the most preferred macrolide azithromycin has been prepared e.g. in example 8 of U.S. Pat. No. 4,474,768. Further to such pre-prepared injectable solutions, solid or pre-dissolved compositions suitable for extemporaneous preparation of solutions immediately prior to administration may advantageously be made from the macrolide. One commonly known marketed example of such a reconstitutable preparation of a macrolide is Zithromax® (azithromycin for injection) by Pfizer. Further to the macrolide and the solvent solutions for injection or the compositions for reconstitution include liquid diluents; for example, propylene glycol, diethyl carbonate, glycerol, sorbitol, etc.; buffering agents, hyaluronidase, local anesthetics and inorganic salts to afford desirable pharmacological properties.

The concentration of the macrolide in the ready-to-use injectable solution may be such that upon use a systemic concentration of about 0,5 to 10 μg/ml serum, preferably about 2 to 5 μg/ml is attained.

The treatments of the invention impede the synthesis of the C₄-HSL and 3-oxo-C₁₂-HSL autoinducers in the P. aeruginosa quorum sensing system by azithromycin at concentrations well below the MIC's of P. aeruginosa, which in turn leads to an efficent suppression of the production of extracellular virulence factors. This opens the way for the prevention of diseases arising from these virulence factors by treatment with macrolides. Moreover, as the 3-oxo-C₁₂-HSL autoinducer has some immunomodulatory activity in itself, stimulating the production of interleukin-8 by respiratory epithelial cells, administration of macrolides to reduce 3-oxo-C₁₂-RSL synthesis might therefore partially prevent the tissue damage arising from chronic inflammatory response in conjection with nosocomial infections.

The invention will be further illustrated by the following examples. These are merely given by way of illustration and are not meant to limit the scope of the appended claims in any way.

### EXAMPLES

### Example 1

### Effect of Azithromycin on Cell growth of P. aeruginosa

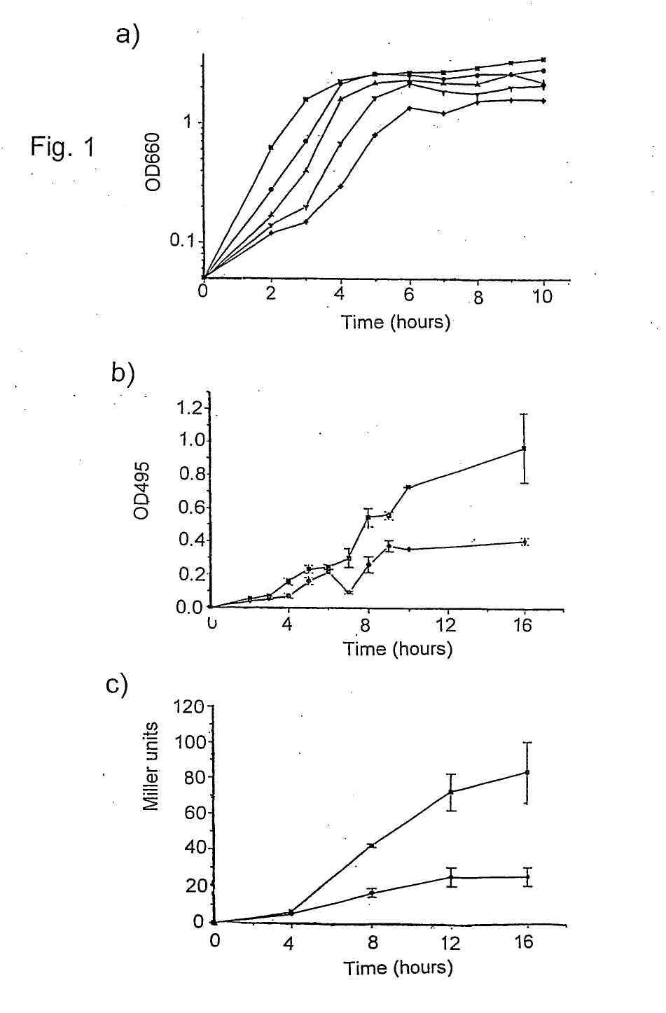

P. aeruginosa strain PAO1 was grown for a total of 10 hours on Luria-Bertani (LB) medium containing 2, 3, 4 and 5 μg/ml of azithromycin, respectively. The cell growth in the media was measured by optical absorbance measurements (turbidity) at 660 nm in intervals of 2 h. The results are shown in FIG. 1a). Exponential growth was slightly affected in the presence of 2 μg of azithromycin/ml, but no effect on the stationary growth phase was observed. Sodium dodecyl sulfate-polyacrylamide gel electrophoresis of total protein extracts of cells grown either in the absence or the presence of 2 μg of azithromycin/ml did not reveal major differences. This experiment shows that an in vitro concentration of azithromcyin of 2 μg/ml does not inhibit PAO1.

### Example 2

### Effect of Azithromycin on Elastase Production

P. aeruginosa strain PAO1 was grown over a total of 16 hours in broth medium either without azithromycin or with 2 μg/ml azithromycin. Samples of the supernatants of both cultures were taken in intervals of 4 hours and the activity of elastase in the samples was determined using elastin Congo red assays (Pearson, J. P., Pesci, E. C., Iglewski, B. H., J. Bacteriol. 1997, 179, 5756-5767). The measured elastase activity was plotted against the sampling times, giving FIG. 1b). The growth curves (data not shown) were also measured by optical density measurements at 660 nm; no significant influence on the growth was observed.

### Example 3

### Effect of Azithromycin on Rhamnolipid Production

P. aeruginosa strain PAO1 was grown on M9-based agar plates (Siegmund, I., Wagner, F., Biotechnol. Tech. 1991, 5, 265-268) into which a gradient of azithromycin from 0 μg/ml to 20 μg/ml was incorporated. The qualitative Rhamnolipid® plate assay was used. The production of rhamnolipids progressively decreased with increasing azithromycin concentrations without a parallel drop in growth.

### Example 4

### Effect of azithromycin on the expression of the rhlAB operon

P. aeruginosa strain PAO1, harbouring the fusion gene pECP60 of rhlA′ with the lacZ reporter (Pesci, E. C., Pearson, J. P., Seed, P. C., Iglewski, B. H., J. Bacteriol. 1997, 179, 3127-3132), was grown for 16 hours in LB medium either in the absence or presence of 2 μg/ml azithromycin and the activity of β-galactosidase (β-Gal) (Miller, J. H., “Experiments in Molecular Genetics”, p. 352-355. Cold Spring Harbor Laboratory, Cold Spring Harbor, N.Y.) was measured in both cultures in intervals of 4 hours. The results of 6 experiments (mean±SD) for each culture were plotted against sampling time (FIG. 1c). The growth curves (data not shown) were also measured by optical density measurements at 660 nm; no significant influence on the growth was observed.

This experiment shows that azithromycin affects, via its interference with autoinducer synthesis, the expression of the rhlAB operon, coding for rhamnosyltransferase (required for rhamnolipid production).

### Example 5

### Effect of Azithromycin on the Expression of the Transcriptional Activator genes lasR and rhlR and on the Expression of the Autoinducer Synthase Genes lasI and rhlI.

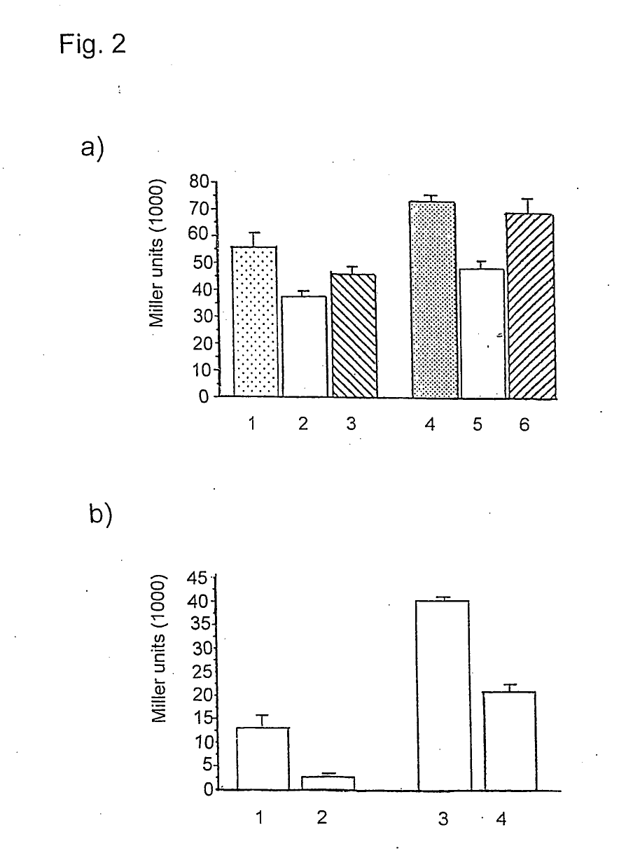

Cultures of P. aeruginosa strain PAO1, harbouring the fusion gene of lasR′ with lacZ reporter (pPCS10011; Pesci, E. C., Pearson, J. P., Seed, P. C., Iglewski, B. H., J. Bacteriol. 1997, 179, 3127-3132), or the fusion gene of rhlR′ with lacZ (pPCS1002; ibid.), or the fusion gene of lasI′ with lacZ (pPCS223; Van Delden, C., Pesci, E. C., Pearson, J. P., Iglewski, B. H., Infect. Immun. 1998, 66, 4499-4502), or the fusion gene of rhlI′ with lacZ (pLPRI; ibid.) were grown for 10 h in broth medium either in the absence or presence of 2 μg/ml azithromycin and the β-Gal activity was then determined. The results for lasR and rhlR are shown in FIG. 2a) and for lasI and rhlI in FIG. 2b) (plotted bars are the mean ±SD of 6 individual experiments each). In the case of lasR and rhlR expression the effect of azithromycin could be almost completely compensated by co-adding to the cultures exogenous 3-oxo-Cl₂—HSL and C₄—HSL in concentrations of 10 μM each (hatched bars in FIG. 2 a). The co-addition of 10 μM exogenous autoinducers could not restore the expression of lasI.

This experiment shows that the interference of azithromycin with the autoinducer synthesis is due to its effect on the transcription of the lasR, rhlR, lasI and rhlI genes.

### Example 6

### Effect of Azithromycin on the Production of 3-oxo-C₁₂-HSL and C₄-HSL Autoinducers

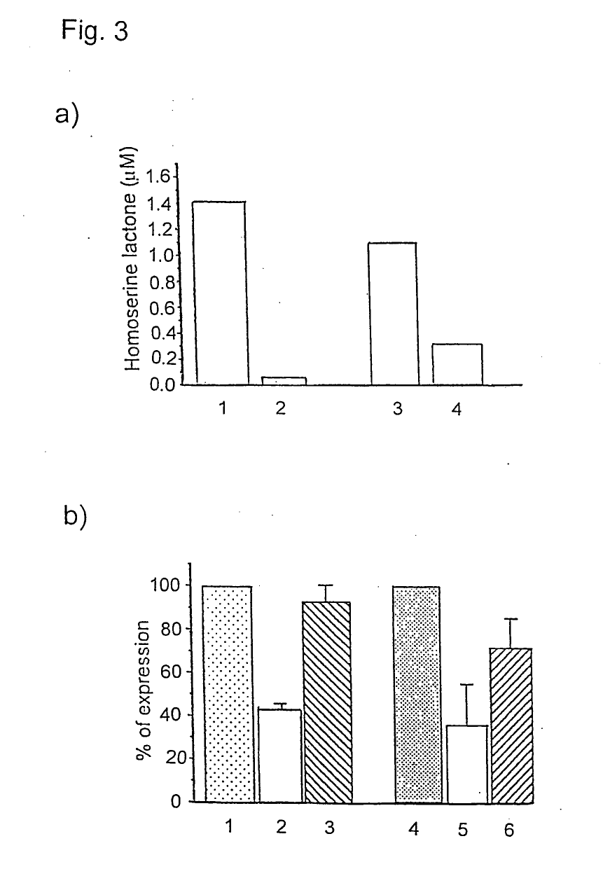

P. aeruginosa strain PAO1 was grown in LB medium for 12 hours either in the absence or presence of 2 μg/ml of azithromycin. The formed 3-oxo-C₁₂—HSL and C₄—HSL autoinducers were extracted from the supernatants of both cultures with ethyl acetate and their respective concentrations were measured using specific bioassays (Pearson, J. P., Pesci, E. C., Iglewski, B. H., J. Bacteriol. 1997, 179, 3127-3132; Seed, P. C, Passador, L., Iglewski, B. H., J. Bacteriol. 1995, 177, 654-659). The results are plotted in FIG. 3 a). In the presence of the macrolide the concentrations of 3-oxo-C₁₂-HSL and C₄-HSL were reduced by 94 and 72%, respectively.

This experiment directly shows the interference of azithromycin with autoinducer synthesis.

### Example 7

### Restoration of rhlAB Expression and Elastase Production by Exogeneous 3-oxo-C₁₂-HSL and C₄-HSL Autoinducers

P. aeruginosa strain PAO1, harbouring the fusion gene pECP60 of rhlA′ with the lacz reporter (Pesci, E. C., Pearson, J. P., Seed, P. C., Iglewski, B. H., J. Bacteriol. 1997, 179, 3127-3132), was grown for 10 hours in LB medium either in the absence or in the presence of 2 μg/ml azithromycin, and, in the latter case, without autoinducers or with 10 mM co-added autoinducers. Both the activity of β-galactosidase (β-Gal) (Miller, J. H., “Experiments in Molecular Genetics”, p. 352-355. Cold Spring Harbor Laboratory, Cold Spring Harbor, NY) and the production of elastase, using elastin Congo red assays (Pearson, J. P., Pesci, E. C., Iglewski, B. H., J. Bacteriol. 1997, 179, 5756-5767)-, was measured in all three cultures after 10 h. The results are shown in FIG. 3b). (plotted bars are the mean±SD of 6 individual experiments each).

### Example 8

### Azithromycin Film-Coated Tablet for Oral Administration

Ingredients (mg/tablet):

| 1) For granulate: | |
|---|---|
| Azithromycin Diydrate USP | 262 |
| (equivalent to 250 mg azithromycin) | |
| Pregelatinized starch | 30 |
| Anhydrous Calcium Phosphate, Dibasic | 100 |
| Sodium croscarmellose | 10 |
| Magnesium stearate/ | 15 |
| Sodium lauryl sulfate 9:1 | |
| 2) Coating: | |
| Eudragit L 30 D-55 ® | 20 |

The ingredients of 1) were wet-granulated using isopropanol as granulating fluid and compressed into tablets using an usual tabletting press. These were then film-coated with 2).

The finished tablet is suited for once-a-day, twice-a-day or thrice-a-day administration, when used in the therapeutic processes according to the invention.

1. A method of treatment or prophylaxis of nosocomial Pseudomonas aeruginosa infections in a subject in need of such treatment or prophylaxis, comprising administering a macrolide antibiotic to said subject in an amount which is effective in impeding quorum sensing in Pseudomonas aeruginosa.

2. The method according to claim 1, wherein the amount is effective in impeding las quorum sensing.

3. The method according to claim 2, wherein the amount is effective in impeding the synthesis of the las quorum sensing autoinducer molecule N-[3-oxododecanoyl]-L-homoserine lactone.

4. The method according to claim 1, wherein the amount is effective in impeding rhl quorum sensing.

5. The method according to claim 4, wherein the amount is effective in impeding the synthesis of the rhl quorum sensing autoinducer molecule N-butyrylhomoserine lactone.

6. The method according to claim 1, whereby the amount is effective in impeding las and rhl quorum sensing.

7. The method according to claim 6, wherein the amount is effective in impeding the synthesis of the las quorum sensing autoinducer molecule N-[3-oxododecanoyl]-L-homoserine lactone and in impeding the synthesis of the rhl quorum sensing autoinducer molecule N-butyrylhomoserine lactone.

8. The method according to claim 1, wherein the macrolide antibiotic is an azalide.

9. The method according to claim 8, wherein the azalide is azithromycin.

10. The method according to claim 1, wherein the infection is selected from the group consisting of ventilator-associated pneumonia in intubated patients, hospital-acquired urinary tract infections, infections in immunocompromised patients, infections in patients with cystic fibrosis, septicemia, pneumonia and chronic inflammatory response in conjection with nosocomial infections.

11. The method according to claim 1, wherein the macrolidep antibiotic is administered intravenously.

12. The method according to claim 1, wherein the macrolide antibiotic is administered orally.

13-25 (canceled).
